(19) United States
(12) Patent Application Publication
(10) Pub. No.: US 2025/0235475 A1
(43) Pub. Date: Jul. 24, 2025

(54) METHOD OF USING MELATONIN LOTION WITH MAGNESIUM TO IMPROVE SLEEP QUALITY AND QUANTITY

(71) Applicant: Proximity Capital Partners LLC, Chicago, IL (US)

(72) Inventor: Stephanie Morimoto, Chicago, IL (US)

(21) Appl. No.: 19/029,613

(22) Filed: Jan. 17, 2025

Related U.S. Application Data
(60) Provisional application No. 63/624,623, filed on Jan. 24, 2024.

(51) Int. Cl. A61K 33/06; A61K 9/00; A61K 9/06; A61K 31/4045; A61P 25/20

(52) U.S. Cl. CPC A61K 33/06; A61K 9/0014; A61K 9/06; A61K 31/4045; A61P 25/20

(57) ABSTRACT

Disclosed herein is a Melatonin Lotion with Magnesium to Improve Sleep Quality and Quantity

### CROSS-REFERENCE TO RELATED APPLICATIONS

The present application claims priority to U.S. provisional application Ser. No. 63/624,623 filed Jan. 24, 2024, herein incorporated by reference in its entirety.

### BACKGROUND

The invention generally relates to dietary supplements that, in combination, help address sleeping issues.

Sleep is a critical factor in maintaining overall health and well-being. Over the past few decades, extensive research has consistently emphasized the pivotal role of sleep in supporting brain function, sustaining physical health, and reducing the risk of various medical conditions [1]. Recent findings have linked sleep deprivation to many detrimental disorders, including hypertension, obesity, type 2 diabetes, compromised immune function, cardiovascular disease, arrhythmias, mood disorders, neurodegenerative conditions, and dementia [2,3]. However, despite this wealth of knowledge, the 2020 Sleep in America poll by the National Sleep Foundation disclosed that more than a third of adults fail to obtain the recommended amount of sleep and experience daytime drowsiness for over half of the week [4,5]. Alarmingly, 55% of those reporting daytime sleepiness attribute it to poor sleep quality [4]. Therefore, exploring methods for enhancing individuals' sleep quality is imperative to bolster their overall physical and mental well-being.

The present invention attempts to solve these problems, as well as others.

### SUMMARY OF THE INVENTION

Provided herein are methods and compositions for a Melatonin Lotion with Magnesium to Improve Sleep Quality and Quantity.

The methods and compositions are set forth in part in the description which follows, and in part will be obvious from the description, or can be learned by practice of the methods and compositions. The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1 are graphs showing the statistical analysis of mean sleep quality and sleep length at Baseline, Day 1, Day 3, Week 1, Week 2, and Week 3. Data is graphed as group means with standard deviation (SD), and individual data points are shown. ns=not significantly different (p=>0.05), *=P<0.05, **=P<0.01, ***=<0.001, ****=P<0.0001. For the sleep quality scale, a decrease in score represents a positive effect of the test product. For the sleep quality score provided by the tracker, an increase represents a positive effect of the test product.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes, or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Description of Embodiments

Generally, the present invention is a sleep composition formulated to target sleep length and quality and method of increasing sleep length and increasing sleep quality by administering an effective amount of the sleep composition. The sleep composition comprises melatonin synergistically working with magnesium in a topical formulation. Melatonin is a hormone naturally synthesized by the body in response to darkness, which plays a pivotal role in regulating the circadian rhythm and facilitating the onset of sleep [6,7]. However, the widespread exposure to artificial light at night poses a challenge, as it can inhibit melatonin production, particularly in our technology-driven society. Numerous studies have demonstrated that oral melatonin supplementation effectively enhances sleep quality and duration [8]. Additionally, magnesium has been researched for sleep regulation and sleep-related health [9]. Magnesium relax the central nervous system, inducing a state of calmness that supports restful sleep [10].

In one embodiment, the sleep composition comprises Water (Aqua), Magnesium Chloride, Dimethyl Sulfone (MSM), Prunus amygdalus dulcis (Sweet Almond) Oil, Butyrospermum parkii (Shea Butter), Sodium Lactate, Cetearyl Alcohol, Glyceryl Stearate Citrate, Glycerin, Undecane, Cetyl Palmitate, Polyglyceryl-3 Diisostearate, Pentylene Glycol, Glyceryl Stearate, Magnesium Sulfate, Melatonin, Tridecane, Potassium Chloride, Sodium Chloride, Calcium Chloride, Tocopheryl Acetate, Xanthan Gum, Hydroxyacetophenone, Sodium Hydroxide, Cymbopogon flexuosus (East-Indian Lemongrass) Oil, Citral, Geraniol, and Linalool.

In the example below, a single-group study evaluated the efficacy of the sleep composition nighttime lotion on sleep length and quality. The method of improving sleep quality comprises applying the sleep composition nightly 30 minutes before bedtime. In one embodiment, the amount of the sleep composition is between about one teaspoon to about two teaspoons to the skin of the subject. In one embodiment, one teaspoon of the sleep composition comprises about 3 mg of melatonin and sleep composition is applied to areas of tension in shoulders, neck, and temples, or on their legs and feet if they experience restless legs or muscle tightness.

In one embodiment, the sleep composition works synergistically to simultaneously upregulate the body's capacity to acclimatize and sleep normally. The sleep composition increased sleep quality with a 45.08% reduction in sleep quality scores by Week 3, where reduction in sleep quality scores means an improvement in sleep quality as indicated below. Additionally, the sleep composition showed significant improvements on Day 3, Week 2, and Week 3. By Week 2 of daily administration, 90.63% of participants showed improvement in sleep quality. By Week 3, 96.88% of daily administration of the sleep composition, participants showed improvement in sleep quality scores.

In one embodiment, the sleep quality improves after at least 2 weeks of treatment by about 90% compared to baseline using a sleep quality scale. In one embodiment, the time asleep improves after at least 2 weeks of treatment by about 60% compared to baseline as tracked by a sleeper tracker. In one embodiment, the sleep quality improves after at least 3 weeks of treatment by about 95% compared to baseline using a sleep quality scale. In one embodiment, the time asleep improves after at least 3 weeks of treatment by about 60% compared to baseline as tracked by a sleeper tracker.

### Formulation

In another aspect, the present invention encompasses a method for treating a sleeping disorder or improving sleep quality in a mammalian subject (preferably, but not exclusively, a mammalian subject) in need of such treatment, wherein said method comprises the administration (systemically, topically or by a combination of routes) of a composition of the present invention.

The present invention further provides a sleep composition for use as medicament or other therapeutic entity (such as ‘herbal remedy’, ‘food supplement’ and the like) in the treatment of an sleep condition. In one embodiment of this aspect, said composition is provided for use as a medicament or other therapeutic entity in the treatment of a systemic or topical.

The present invention further provides the use of a sleep composition as disclosed herein for the preparation of a medicament. In some embodiments, this aspect of the invention relates to the use of a topical formulation as disclosed herein in the preparation of a medicament for use in the treatment of an sleep disorder or improve sleep quality.

In certain embodiments, the dosage form is formulated as granules, pellets, micro particles, tablet, hard shell capsules, suspended in a liquid, suspended in a syrup or enema. In certain embodiments, the dosage form is formulated for oral or mucosal delivery. In certain embodiments, the dosage form is formulated as or in a lozenge, candy, toffee, chocolate or cookie. In certain embodiments, the tablet or pellets are an immediate release or slow or controlled release dosage forms. In certain embodiments, the tablet is enteric coated or is a melt or dissolved in the mouth or is muco-adhesive dosage form.

In certain embodiments, the unit dosage form which is a unit particles, such as tablet, capsule, granules, pellets, micro-particles and film, are enteric coated or coated with a colonic coat that protect the unit dose from being decomposed at the acidic gastric pH and swells in time manner of pH controlled manner or both, to release the sleep composition at the distal intestine and may also release part of the sleep composition in the intestines for systemic absorption and part of the Sleep composition for pharmacological effect.

In certain embodiments, the sleep composition is formulated in a semi solid or liquid dosage form such as cream, lotion, ointment, dispersion, suspension, gel, foam, spray, syrup, liquid, eye drops, ear drops, enema or an oral dosage form or a topical dosage form or a local ophthalmic or optic or oral cavity or vaginal or rectal or uterine dosage form.

In certain embodiments, any one of the compositions described above, or any one of the dosage forms described above, is for use in a method of treating sleeping disorders or improving sleep quality. Preferred dosage forms include, but are not limited to, any liquid or semi solid or solid dosage form. The composition may be formulated in a medicament by preparing a topical or mucosal or oral delivery system. The topical delivery system may be in form of eye drops, a suspension, ointment, cream, foam, spray, topical patch. The oral delivery system may be a tablet or capsule or soft capsule or sachet or granules or a syrup. The mucosal delivery system may be a gel, pessary, enema, douche, wash, foam, mucoadhesive gel or tablet for immediate or for slow or controlled release. The vehicle may comprise any acceptable solvent and inactive ingredients as well as preservatives anti-oxidants and coloring agents. The delivery form may be single dose or multiple dose as well as micro particle granulate nanoparticle microcapsule liposome micelle, and the like as known in the art of pharmaceutical, cosmetic, veterinary medicine and art of formulation. Further details of suitable dosage forms may be obtained from any standard reference work in this field, including, for example: Remington's Pharmaceutical Sciences, Mack Publishing Co, Easton, Pa, USA (1980).

Thus, in some embodiments of the present invention, the composition further comprises one or more excipients selected from the group consisting of solvents, stabilizers, suspending agents, emulsifiers, release modifying, targeting and viscosity agents and combinations thereof.

In some embodiments, the composition of the present invention is formulated as a dosage form selected from the group consisting of a liquid, a suspension, an emulsion, a foam, a spray, a liposome, a semi-solid, a cream, an ointment, a patch, a particulate formulation, a granulate, a micro-particulate formulation, a nano-particulate formulation, a solid dosage form, a tablet, a capsule, an orally-disintegrable capsule, a mouth wash and an adhesive buccal tablet.

According to particular embodiments, the compounds or derivatives prepared according to embodiments of the methods of the present disclosure can comprise compounds or derivatives, or salts, hydrates, solvates, or prodrugs thereof, or crystalline forms thereof, substantially free of solvents or other by-products, generally, or a particular solvent or by-product. In certain embodiments, by “substantially free” is meant greater than about 80% free of solvents or by-products, or greater than about 80% free of a particular solvent or by-product, more preferably greater than about 90% free of solvents or by-products, or greater than about 90% free of a particular solvent or by-product, even more preferably greater than about 95% free of solvents or by-products, or greater than about 95% free of a particular solvent or by-product, even more preferably greater than 98% free of solvents or by-products, or greater than about 98% free of a particular solvent or by-product, even more preferably greater than about 99% free of solvents or by-products, or greater than about 99% free of a particular solvent or by-product, even more preferably greater than about 99.99% free of solvents or by-products, or greater than about 99.99% free of a particular solvent or by-product, and most preferably quantitatively free of solvents or by-products, or quantitatively free of a particular solvent or by-product.

For preparing pharmaceutical compositions from a Sleep composition form or a hydrate, solvate, or prodrug thereof, prepared according to the methods of the present disclosure, pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active components. In tablets, the active component is mixed with the carrier having the necessary binding capacity in suitable proportions and compacted in the shape and size desired.

The powders and tablets preferably contain from about 1 to about 99.99 percent of the active form of Sleep composition, or salt, hydrate, solvate, or prodrug thereof, prepared according to the methods of the present disclosure. Suitable carriers are microcrystalline cellulose, sugar, lactose, pectin, dextrin, starch, gelatin, tragacanth, methylcellulose, sodium carboxymethylcellulose, a low melting wax, cocoa butter, and the like, and other excipients may include magnesium stearate, stearic acid, talc, silicon dioxide, etc. Dosages of the active form of Sleep composition may be between about 10 mg to about 10000 mg in the preparation for example. The term “preparation” is intended to include the formulation of active compound with encapsulating material as carrier providing a capsule in which the active component, with or without carriers, is surrounded by a carrier, which is thus in association with it. Tablets, powders, capsules, pills, sachets, and lozenges are included. Tablets, powders, capsules, pills, sachets, and lozenges can be used as solid forms suitable for oral administration. Liquid preparations include solutions, suspensions, and emulsions, for example, water or water-propylene glycol solutions. For example, parenteral injection liquid preparations can be formulated as solutions in aqueous polyethylene glycol solution. The crystalline forms of Sleep composition extracts, or salts, hydrates, solvates, or prodrugs thereof prepared according to the methods of the present disclosure may thus be formulated for parenteral administration (e.g., by injection, for example bolus injection or continuous infusion) and may be presented in unit dose for example in ampoules, pre-filled syringes, small volume infusion, or in multi-dose containers with an added preservative). The compositions may take such forms as suspensions, solutions, or emulsions in oily or aqueous vehicles, and may contain formulation agents such as suspending, stabilizing, and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use. The method of administration may be via inhalation and topical routes. Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Compositions suitable for topical administration include the active ingredient in an inert base such as gelatin and glycerine or sucrose and acacia; and mouthwashes comprising the active ingredient in suitable liquid carrier.

Solutions or suspensions are applied directly to the skin by conventional means, for example with a dropper, pipette, or spray. The compositions may be provided in single or multi-dose form. In compositions intended for administration to the respiratory tract, including intranasal compositions, the compound or derivative will generally have a small particle size, for example on the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets, capsules, tinctures, and lozenges for oral administration and liquids for oral use are preferred compositions. Solutions or suspensions for application to the nasal cavity or to the respiratory tract are preferred compositions. Transdermal patches for topical administration to the epidermis are preferred compositions.

Further details on techniques for formulation and administration may be found in the latest edition of REMINGTON'S PHARMACEUTICAL SCIENCES (Mack Publishing Co., Easton, PA).

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Further details on techniques for formulation may be found in the latest edition of Remington's Pharmaceutical Sciences (Mack Publishing Co., Easton, PA).

### Definitions

The term “administration” or “administering” refers to a method of providing a dosage of a compound or active ingredient or pharmaceutical composition to a subject, where the method is epicutaneous (topical) or subcutaneous. Modes of administration, dosing schedules disclosed compounds and compositions can be determined according to the criteria generally taken into account in the establishment of a sleep disorder treatment adapted to, for example, a patient's sleep pattern. The compositions can be administered such that they conditioned to be treated before falling asleep.

In the present disclosure, an “effective amount” or an “effective dose” of the sleep composition, or composition refers to an amount of sleep composition that, once administered to a subject, will reach the subject's bloodstream and/or bodily tissues.

The term “pharmaceutical composition” as used herein has its conventional meaning and refers to a composition which is pharmaceutically acceptable. The term “pharmaceutically acceptable” as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. Pharmaceutical composition includes configurational isomers (such as cis and trans isomers) and all optical isomers (such as enantiomers) Isomers and diastereomers), racemic, diastereoisomers and other mixtures of these isomers, as well as solvates, hydrates, isomorphs, polymorphs, tautomers, Ester, salt forms and prodrugs. The term “prodrug” refers to a compound that is a drug precursor, which releases the drug in vivo through some chemical or physiological processes after administration (for example, the prodrug is transformed into the desired drug form when it reaches physiological pH or through the action of enzymes). Exemplary prodrugs release the corresponding free acid upon cleavage, and the hydrolyzable ester-forming residues of the compounds of the present invention.

The term “pharmaceutically acceptable” refers to derivatives, analogues and salts which are physiologically acceptable for use in mammals, and which are not unduly toxic or otherwise unacceptable for such use. The term “mammals” includes human and non-human mammals, including domestic animals, e.g. cats, dogs, rodents, cattle, horses and the like, as well as non-domesticated animals.

The term “excipient” as used herein has its conventional meaning and refers to a pharmaceutically acceptable ingredient, which is commonly used in the pharmaceutical technology for preparing a granulate, solid or liquid oral dosage formulation. The term “cosmetic composition” is intended to mean a substance or a preparation intended to be brought into contact with the various superficial parts of the body, in particular the epidermis, the body-hair and head-hair systems, the nails, the lips and the oral mucous membranes. The term “veterinary composition” encompasses the full range of compositions for internal administration and feeds and drinks which can be consumed by animals.

As used herein, the term “solvent” refers to a compound or mixture of compounds including, but not limited to, water, water in which an ionic compound has been dissolved, acetic acid, acetone, acetonitrile, benzene, 1-butanol, 2-butanol, t-butyl alcohol (“TBA”), 2-butanone, carbon tetrachloride, chlorobenzene, chloroform, cyclohexane, 1,2-dichloroethane (“DCE”), diethylene glycol, diethyl ether (“Et₂O”), diglyme (diethylene glycol dimethyl ether), 1,2-dimethoxyethane (“DME”), N,N-dimethylformamide (“DMF”), dimethylsulfoxide (“DMSO”), 1,4-dioxane, ethanol, ethyl acetate (“EtOAc”), ethylene glycol, glycerin, heptanes, hexamethylphosphoramide (“HMPA”), hexamethylphosphorus triamide (“HMPT”), hexane, methanol (“MeOH”), methyl t-butyl ether (“MTBE”), methylene chloride (“DCM,” “CH₂Cl₂”), N-methyl-2-pyrrolidinone (“NMP”), nitromethane, pentane, petroleum ether, 1-propanol (“n-propanol,” “n-PrOH”), 2-propanol (“isopropanol,” “iPrOH”), pyridine, tetrahydrofuran (“THF”), toluene, tributylamine, triethylamine (“TEA,” “Et₃N”), o-xylene, m-xylene, and/or p-xylene, and the like. Solvent classes may include hydrocarbon, aromatic, aprotic, polar, alcoholic and mixtures thereof.

The term “synergistic” as used herein is refers to the phenomenon wherein the cumulative pharmacological effect of two or more ingredients when used in combination is higher than the sum of the effect of each of them tested individually. The term “potentiating” as used herein refers to the phenomenon where the efficacy of an active ingredient is significantly enhanced when it is combined with a second ingredient, wherein said second ingredient itself does not demonstrate any efficacy in the same pharmacological test. In some cases of potentiation, not only is said second ingredient devoid of the pharmacological effect being measured, it may even cause an opposite effect, when assayed alone. An example of such a case would be as follows: ingredient A is anti-inflammatory; ingredient B is pro-inflammatory; when A and B are combined, said combination produces an anti-inflammatory effect that is greater than seen with A alone. In the context of the present invention, potentiation is regarded as a special case of synergism. Thus, the term ‘synergism’ (or synergistic, or the like), when used to define the properties of a composition of the present invention, also includes within its range of meaning the potentiation effect described immediately hereinabove.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: A Single-Group Study to Examine the Efficacy of the Sleep Composition to Improve Sleep Quality and Quantity

### Methods

A total of 35 participants aged 18-59 were recruited for this study. Three participants were removed for inactivity. Participant demographic information is listed in Table 1. All participants satisfied the following inclusion and exclusion criteria.

TABLE 1
Participant Demographic Information

| Ethnicity | N | % |
|---|---|---|
| White | 19 | 59.38 |
| Hispanic or Latino | 4 | 12.5 |
| Black or African-American | 3 | 9.38 |
| Asian | 6 | 18.75 |

Inclusion criteria is as follows: 1) Male or female aged 18-59; 2) Must agree to not use any other sleep aids, including prescriptions, supplements, or melatonin products, during the study or 28 days prior to Baseline; 3) Has favorable sleep conditions (a dark, quiet room to sleep in and is not woken up regularly throughout the night due to personal circumstances, e.g., by a baby); 4) Willing to maintain their standard sleep pattern and activity level for the duration of the study; 5) Be generally healthy and not living with any uncontrolled chronic disease; and 6) Willing and able to supply and use their own sleep tracking device.

Exclusion criteria is as follows: 1) Diagnosed with any chronic sleep condition including insomnia, narcolepsy, or sleep apnea; 2) Has extremely sensitive/irritable skin or diagnosed skin conditions; 3) Has poor sleep hygiene as a result of specific personal circumstances e.g., new mothers; 4) Women who are pregnant, breastfeeding, or trying to conceive; 5) Anyone unwilling or unable to follow the study protocol; 6) Any pre-existing chronic conditions that would prevent participants from adhering to the protocol, including oncological and psychiatric disorders; 7) Is currently undergoing, or planning to undergo any sleep-related procedures in the next three weeks; 8) A history of severe allergic reactions including but not limited to any of the product's ingredients; and 9) Heavy drinkers or drug users. A heavy drinker is considered to be a woman who drinks eight or more alcoholic drinks per week or a man who drinks 15 or more alcoholic drinks per week.

### Study Design

This was a virtual study that required participants to complete questionnaires at home and wear a sleep tracker. Consent forms describing the study process, instructions, evaluation methods, and bill of rights were provided to participants before study onboarding. Following the consent process, participants completed the baseline survey evaluating overall sleep quality and wore the sleep tracker for one week before the study started to collect sleep tracking information.

The questionnaire used was a modified version of the Sleep Quality Scale [11].

Participants were then instructed to use the product per the sponsor's specific guidance:

Participants will begin applying the lotion nightly 30 minutes before bedtime and use a sleep tracker nightly.

Participants should apply two teaspoons (one teaspoon of lotion covers approximately the size of the fingertip from tip to first knuckle) of lotion (3 mg of melatonin) and use it wherever they would usually use body lotion (paying particular attention to areas of tension in shoulders, neck, and temples, or on their legs and feet if they experience restless legs or muscle tightness).

Participants completed questionnaires and provided sleep tracker results at Baseline, Day 1, Day 3, Week 1, Week 2, and Week 3.

### Data Analysis

Data were collected using a textual 4-point or 5-point Likert scale for each question. The sleep quality scale used a textual 4-point scale for each question from “Rarely” to “Almost Always”. The textual Likert data was transformed into numerical values of 0-3, with 0 representing the most favorable/best outcome (“Rarely”) and 3 representing the least favorable/worst outcome (“Almost Always”). This was reversed for questions relating to positive aspects (“I feel refreshed after sleep”). The score was then tallied for an overall sleep quality score, with a higher score representing worse sleep quality.

Data were checked for normality using the Pearson test. A repeated measure analysis compared participant outcomes at each check-in to their Baseline response. Data were analyzed using the repeated measure one-way ANOVA or Friedman Tests based on the normality of the data. Statistical analyses were performed in GraphPad Prism 9.0, and the significance level was set at 0.05. For product-specific questions evaluated only on Day 1, Day 3, Week 1, Week 2, and

Week 3, results were presented as % of subjects reporting each answer.

### Data Interpretation

By Day 3, the sleep composition (Asutra nighttime lotion) significantly improved: Overall sleep quality, Sleep length. By Week 3, sleep composition (Asutra nighttime lotion) significantly improved: Overall sleep quality, Tracked sleep quality, and Sleep length

### By Week 3:

78.1% of participants found that the lotion felt nice on their skin
71.9% had a better night's sleep after using the lotion
71.9% agreed that their sleep quality was better after using the lotion
71.9% of participants would like to continue using the lotion
71.9% of participants would recommend the lotion to a family or friend
68.8% of participants found that they would fall asleep more easily and quickly after using the lotion
65.6% of participants found that they fell back to sleep easier or more quickly when waking up in the night after using the lotion
62.5% of participants found that they felt more rested after using the lotion
62.5% of participants found that they would purchase the lotion

### Overall:

90.63% of participants experienced an improvement in self-reported sleep quality scores by Week 2.
96.88% of participants experienced an improvement in self-reported sleep quality scores by Week 3.

### Results

### 3.1 the Sleep Composition (Asutra Nighttime Lotion) Significantly Improves Sleep Quality and Length Over Time

All subjects self-scored their overall sleep quality on a 4-point textual Likert scale at the Baseline, Day 1, Day 3, Week 1, Week 2, and Week 3. The textual Likert data was transformed into numerical values of 0-3. Statistical analysis compared the overall sleep quality score for each patient at each check-in to the Baseline measure.

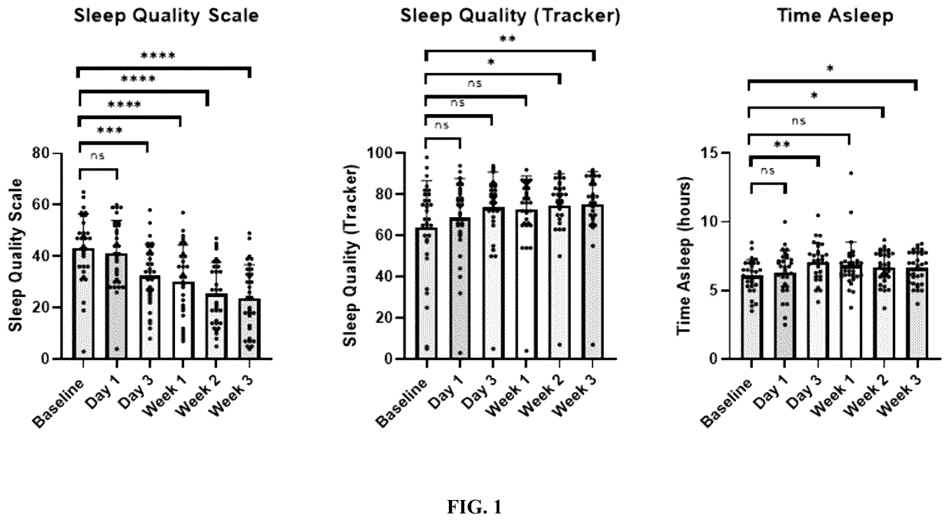

By Day 3, the Sleep Quality Scale showed significant improvements (a reduction in score), consistent throughout the study. By Week 3, there was a total 45.08% reduction in the score, where the reduction in sleep quality scores means an improvement in sleep quality (Table 2, FIG. 1). Notably, at Week 2, 90.63% of participants showed improvement in sleep quality. By Week 3, 96.88% of participants showed improvement in sleep quality scores (Table 4).

TABLE 2
Statistical outcomes of overall health and wellbeing parameters changes.
% Change indicates a change in mean values from baseline to endline.
Green cells highlight statistically significant outcomes.

| | | Sleep | Sleep Quality | |
|---|---|---|---|---|
| | | Quality Scale | (Tracker) | Time Asleep |
| Baseline | Mean | 43.19 | 63.97 | 6.07 |
| | STD | 13.24 | 22.64 | 1.17 |
| Day 1 | Mean | 41.16 | 68.81 | 6.35 |
| | STD | 12.84 | 19.02 | 1.57 |
| | P-value | 0.4448 | >0.9999 | >0.9999 |
| Day 3 | Mean | 31.66 | 74.00 | 7.01 |
| | STD | 12.24 | 17.40 | 1.25 |
| | P-value | 0.0005 | 0.1495 | 0.0029 |
| Week 1 | Mean | 30.34 | 72.88 | 6.64 |
| | STD | 13.96 | 16.21 | 1.22 |
| | P-value | <0.0001 | 0.2633 | 0.1373 |
| Week 2 | Mean | 25.41 | 74.78 | 7.72 |
| | STD | 12.29 | 15.46 | 5.44 |
| | P-value | <0.0001 | 0.0147 | 0.0416 |
| Week 3 | Mean | 23.72 | 75.44 | 6.65 |
| | STD | 13.07 | 15.73 | 1.18 |
| | P-value | <0.0001 | 0.0095 | 0.0251 |
| | % | −45.08 | 17.93 | 9.48 |
| | Change | | | |

The sleep scores from the tracker did not show such immediate improvements; however, by Week 2, a significant improvement was observed, consistent through to Week 3 (Table 2, FIG. 1).

TABLE 4
Percentage of participants who experienced an
improvement in scores compared to the Baseline.

| % Participants showing | | | |
|---|---|---|---|
| Improvement compared to | Sleep Quality | Sleep Quality | Time |
| Baseline | Scale | (Tracker) | Asleep |
| Week 2 | 90.63% | 68.75% | 62.50% |
| Week 3 | 96.88% | 59.38% | 59.38% |

The tracker also measured the time spent sleeping per night. The mean time slept was then calculated. Some variation was observed in these results, with significant improvements seen on Day 3, non-significant improvements on Week 1, and then significant improvements were observed again on Week 2 and Week 3 (Table 2, FIG. 1).

3.2 the Sleep Composition (Asutra Nighttime Lotion) was Well-Received, with Positive Results

Participants were asked to respond to questions on Day 1, Day 3, Week 1, Week 2, and Week 3. These questions provided insight into the participant's perception of the product and how it improves sleep quality. Participants responded to product evaluation questions on a “strongly disagree” to “strongly agree” scale. The “strongly agree” and “agree” responses were combined into a single “combined agree” or similar outcomes to better evaluate the overall agreement.

The participants responded positively to the product, with 5 out of 14 showing a combined agree score of over 70%. Regarding improvement in sleep quality, the highest scores were 71.9%. These were “I had a better night's sleep after using the lotion” and “My sleep is better quality after using the lotion”. Further scores over 70% included the 78.1% of participants who agreed that the lotion felt nice on their skin and 71.9% who would continue using the lotion and recommend it to a family member or friend (Table 3).

TABLE 3
Participant response scores to product-specific questions

| | | | Week | Week | Week |
|---|---|---|---|---|---|
| Question | Day 1 | Day 3 | 1 | 2 | 3 |
| How much do you agree or disagree with the | Combined Agree % | | | | |
| following statement: | | | | | |
| I had a better night's sleep after using the lotion. | 40.6% | 65.6% | 71.9% | 65.6% | 71.9% |
| My sleep is better quality after using the lotion. | 40.6% | 62.5% | 71.9% | 65.6% | 71.9% |
| I sleep for longer after using the lotion. | 43.8% | 46.9% | 56.3% | 53.1% | 53.1% |
| I feel more rested the next day after using the lotion. | 37.5% | 56.3% | 62.5% | 68.8% | 62.5% |
| I fell asleep more easily and/or quickly after using | 53.1% | 59.4% | 62.5% | 68.8% | 68.8% |
| the lotion. | | | | | |
| I fell back to sleep easier or more quickly when | 46.9% | 53.1% | 62.5% | 71.9% | 65.6% |
| waking up in the night and using the lotion. | | | | | |
| I had more energy the next day after using the lotion. | 31.3% | 40.6% | 50.0% | 59.4% | 53.1% |
| I felt more able to complete day-to-day tasks the next | 37.5% | 50.0% | 56.3% | 68.8% | 59.4% |
| day after using the lotion. | | | | | |
| The lotion felt nice on my skin. | 75.0% | 71.9% | 71.9% | 84.4% | 78.1% |
| Compared to my usual melatonin product, I wake up | 53.1% | 43.8% | 62.5% | 62.5% | 53.1% |
| feeling less groggy after using this product. | | | | | |
| I would like to continue using the lotion. | | | | | 71.9% |
| I would purchase the lotion. | | | | | 62.5% |
| I would recommend the lotion to a family member or | | | | | 71.9% |
| friend. | | | | | |

Combined Not at all/A little bit

| | | | | | |
|---|---|---|---|---|---|
| After using this product, how groggy do you feel | 68.8% | 68.8% | 65.6% | 75% | 75% |
| upon waking? | | | | | |

Participants' perception of grogginess upon waking also improved over time, with more participants reporting that they did not experience grogginess at all or a little bit (68.8% on Day 1 compared to 75% on Week 3). However, only 53.1% reported that compared to their usual melatonin product, they felt less groggy (Table 3).

### Discussion

The results of this study indicate that the sleep composition (Asutra nighttime lotion) has a significant and positive impact on sleep quality and duration over time. Participants' self-reported assessments of their sleep quality showed consistent improvement throughout the study, with a 45.08% reduction in sleep quality scores by Week 3, where the reduction in sleep quality scores means an improvement in sleep quality. Additionally, while there were variations in the time spent sleeping per night, significant improvements were observed on Day 3, Week 2, and Week 3. The variations could be attributed to measurement errors or variations between sleep times depending on the night.

These findings align with the hypothesis that the sleep composition (Asutra nighttime lotion) may lead to tangible and sustained enhancements in participants' sleep experiences. Notably, the positive effects on sleep quality were observed both through self-reported sleep quality scores and tracker-measured data. It is essential to highlight the significance of these improvements, as sleep quality is intricately linked to overall well-being, and even slight enhancements can lead to a better quality of life.

Participants' feedback gathered over multiple time points revealed reasonable satisfaction with the product. Most participants strongly agreed with statements such as “I had a better night's sleep after using the lotion” and “My sleep is better quality after using the lotion”. Furthermore, participants reported that the lotion felt pleasant on their skin, and a significant proportion expressed their intention to continue using it and recommend it to others.

Another noteworthy aspect of the study is the reduction in grogginess reported by participants upon waking. Throughout the study, an increasing number of participants reported experiencing little to no grogginess in the morning, a positive indicator of improved sleep quality. If the study continued, then this might further improve.

While these findings are promising, it is important to acknowledge some study limitations. The study design relied on self-reported data and lacked a control group, which can introduce potential bias and make it challenging to establish a causal relationship between the lotion and the observed improvements. More extensive clinical research studies, including gold-standard randomized-controlled trials, would be recommended to further establish the efficacy of sleep composition (Asutra nighttime lotion).

### CONCLUSION

In conclusion, the results of this study suggest that the sleep composition (Asutra nighttime lotion) showed significant improvements in sleep quality and duration and reduced morning grogginess. The positive feedback from participants indicates a high level of user satisfaction. These findings provide a strong foundation for future research and support the potential use of the sleep composition (Asutra nighttime lotion) to enhance sleep quality and overall well-being.

### REFERENCES

1. Worley S L. The Extraordinary Importance of Sleep. P T. 2018; 43 (12): 758-763.
2. Colten H R, Altevogt B M, Research I of M (U S) C on S M and. Extent and Health Consequences of Chronic Sleep Loss and Sleep Disorders. In: Sleep Disorders and Sleep Deprivation: An Unmet Public Health Problem. National Academies Press (U S); 2006. Accessed Oct. 19, 2023. https://www.ncbi.nlm.nih.gov/books/NBK19961/
3. Sleep Deprivation and Deficiency-What Are Sleep Deprivation and Deficiency? | NHLBI, 24, 2022. Accessed Oct. 19, 2023. NIH. Published March https://www.nhlbi.nih.gov/health/sleep-deprivation
4. 2020 Sleep in America® Poll Shows Alarming Level of Sleepiness and Low Levels of Action. National Sleep Foundation. Published Mar. 9, 2020. Accessed Oct. 19, 2023. https://www.thensf.org/2020-sleep-in-America-poll-shows-alarming-level-of-sleepiness/
5. Sleep by the Numbers. National Sleep Foundation. Published May 12, 2021. Accessed Oct. 19, 2023. https://www.thensf.org/sleep-facts-and-statistics/
6. Tordjman S, Chokron S, Delorme R, et al. Melatonin: Pharmacology, Functions and Therapeutic Benefits. Curr Neuropharmacol. 2017; 15 (3): 434-443. doi: 10.2174/1570159X14666161228122115
7. Zisapel N. New perspectives on the role of melatonin in human sleep, circadian rhythms and their regulation. Br J Pharmacol. 2018; 175 (16): 3190-3199. doi: 10.1111/bph.14116
8. Costello R B, Lentino C V, Boyd C C, et al. The effectiveness of melatonin for promoting healthy sleep: a rapid evidence assessment of the literature. Nutr J. 2014; 13:106. doi: 10.1186/1475-2891-13-106
9. Arab A, Rafie N, Amani R, Shirani F. The Role of Magnesium in Sleep Health: a Systematic Review of Available Literature. Biol Trace Elem Res. 2023; 201 (1): 121-128. doi: 10.1007/s12011-022-03162-1
10. Schwalfenberg G K, Genuis S J. The Importance of Magnesium in Clinical Healthcare. Scientifica (Cairo). 2017; 2017:4179326. doi: 10.1155/2017/4179326
11. Shahid A, Wilkinson K, Marcu S, Shapiro C M. Sleep Quality Scale (SQS). In: Shahid A, Wilkinson K, Marcu S, Shapiro C M, eds. STOP, THAT and One Hundred Other Sleep Scales. Springer New York; 2011:345-350. doi: 10.1007/978-1-4419-9893-4_85

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of treating a sleeping disorder in a subject, comprising: administering an effective amount of a sleep composition to the subject, wherein the sleep composition includes melatonin and magnesium.

2. The method of claim 1, wherein the sleep composition is in lotion form.

3. The method of claim 2, wherein the administering step comprises applying the lotion form at least 30 minutes before bedtime.

4. The method of claim 3, wherein the lotion form comprises at least two teaspoons and about 3 mg of melatonin.

5. The method of claim 4, wherein the lotion form comprises Water, Magnesium Chloride, Dimethyl Sulfone, Prunus amygdalus dulcis Oil, Butyrospermum parkii, Sodium Lactate, Cetearyl Alcohol, Glyceryl Stearate Citrate, Glycerin, Undecane, Cetyl Palmitate, Polyglyceryl-3 Diisostearate, Pentylene Glycol, Glyceryl Stearate, Magnesium Sulfate, Melatonin, Tridecane, Potassium Chloride, Sodium Chloride, Calcium Chloride, Tocopheryl Acetate, Xanthan Gum, Hydroxyacetophenone, Sodium Hydroxide, Cymbopogon flexuosus Oil, Citral, Geraniol, and Linalool.

6. The method of claim 5, wherein the sleep quality improves after at least 2 weeks of treatment by about 90% compared to baseline using a sleep quality scale.

7. The method of claim 6, wherein the time asleep improves after at least 2 weeks of treatment by about 60% compared to baseline as tracked by a sleeper tracker.

8. The method of claim 5, wherein the sleep quality improves after at least 3 weeks of treatment by about 95% compared to baseline using a sleep quality scale.

9. The method of claim 8, wherein the time asleep improves after at least 3 weeks of treatment by about 60% compared to baseline as tracked by a sleeper tracker.

10. A composition or formulation for improving sleep quality, comprising a sleep composition for treating a sleeping disorder, wherein the sleep composition includes melatonin and magnesium.

11. The composition of claim 10, wherein the composition or formulation is in lotion form.

12. The composition of claim 11, wherein the composition or formulation comprises Water, Magnesium Chloride, Dimethyl Sulfone, Prunus amygdalus dulcis Oil, Butyrospermum parkii, Sodium Lactate, Cetearyl Alcohol, Glyceryl Stearate Citrate, Glycerin, Undecane, Cetyl Palmitate, Polyglyceryl-3 Diisostearate, Pentylene Glycol, Glyceryl Stearate, Magnesium Sulfate, Melatonin, Tridecane, Potassium Chloride, Sodium Chloride, Calcium Chloride, Tocopheryl Acetate, Xanthan Gum, Hydroxyacetophenone, Sodium Hydroxide, Cymbopogon flexuosus Oil, Citral, Geraniol, and Linalool.
